(19) United States
(12) Patent Application Publication
(10) Pub. No.: US 2015/0105324 A1
(43) Pub. Date: Apr. 16, 2015

(54) COMPOSITIONS AND METHODS FOR TREATING PTSD AND RELATED DISEASES

(71) Applicant: INDIANA UNIVERSITY RESEARCH AND TECHNOLOGY CORPORATION, Indianapolis, IN (US)

(72) Inventors: Anantha Shekhar, Indianapolis, IN (US); Yvonne Lai, Bloomington, IN (US)

(21) Appl. No.: 14/397,386

(22) PCT Filed: Apr. 26, 2013
(86) PCT No.: PCT/US13/38443

§ 371 Date: Oct. 27, 2014

Related U.S. Application Data
(60) Provisional application No. 61/639,687, filed on Apr. 27, 2012.

(51) Int. Cl. C07K 14/47; C07K 5/11; C07K 7/06; A61K 38/17; A61K 31/606; A61K 31/445; A61K 38/16; C07K 5/113; A61K 31/4192

(52) U.S. Cl. CPC C07K 14/47; C07K 5/1021; C07K 5/1019; C07K 7/06; A61K 31/4192; A61K 31/606; A61K 31/445; A61K 38/162; A61K 38/1787
USPC 514/17.3; 514/359; 514/166; 514/317

(57) ABSTRACT

The invention described herein pertains to compositions and methods for treating PTSD and related diseases. In particular, the invention described herein pertains to compositions and methods for treating PTSD and related diseases by administering modulators of NMDA NR2-PSD95-nNOS signaling.

### CROSS REFERENCE TO RELATED APPLICATIONS

This application claims priority under 35 U.S.C. §119(e) to U.S. Provisional Application Ser. No. 61/639,687, filed Apr. 27, 2012, the disclosure of which is incorporated herein in its entirety.

### INCORPORATION BY REFERENCE OF MATERIAL SUBMITTED ELECTRONICALLY

Incorporated by reference in its entirety is a computer-readable sequence listing submitted concurrently herewith and identified as follows: one 4,453 bytes ASCII (text) file named “225045_ST25.TXT,” created on Apr. 26, 2013.

### TECHNICAL FIELD

The invention described herein pertains to compositions and methods for treating PTSD and related diseases. In particular, The invention described herein pertains to compositions and methods for treating PTSD and related diseases by administering modulators of NMDA NR2-PSD95-nNOS signaling.

### BACKGROUND AND SUMMARY OF THE INVENTION

Post traumatic stress disorder (PTSD) is a severe anxiety disorder that develops following exposure to a traumatic event. It is frequently accompanied by other co-morbid psychiatric and medical illnesses along with high rates of functional disability [1-4]. In the general population, the lifetime prevalence rate of PTSD is approximately 8-10% [1] [2], but this could increase to as high as 20% following chronic stressor such as combat exposure [3]. Although serotonin reuptake inhibitors are the current first-line medications for treatment of PTSD symptoms, less than 60% of subjects get only partial benefits from them [4], highlighting a great unmet need to develop novel therapeutics for this population. The commonly accepted pathophysiology model suggests that PTSD develops in a subset of trauma exposed subjects, at least in part, due to enhanced conditioned fear to trauma-associated cues. In this model, PTSD symptoms develop when a traumatic event (unconditioned aversive stimulus, US) is paired with a variety of non-aversive conditioned stimuli (CS) causing persistent conditioned fear, and characteristic deficits in the extinction of those conditioned fear responses are also observed.

Fear conditioning processes have been reported to be dependent on activation by glutamate of N-methyl-D-aspartic acid receptors (NMDAR) and its various downstream signaling mechanisms that result in long-term plasticity within a neural network comprising of key structures such as the amygdala, prefrontal cortex and hippocampus [5, 6]. One such downstream effect following NMDAR stimulation involves activation of neuronal nitric oxide synthase (nNOS). Activation of NMDAR by glutamate stimulates nNOS, which is coupled to the scaffolding protein postsynaptic density protein 95 (PSD95), resulting in NO production [9]. The production of NO has been implicated in consolidation of conditioned fear using both pharmacological and gene knock out methods [7-13]. In conditioned fear models, NO appears to be a retrograde signal at presynaptic terminals of the amygdala [14, 15], where acting through guanylyl cyclase and cGMP-dependent protein kinase (PKG)[10], it increases transcription of immediate early genes c-Fos and Erg-1 [16-19], molecules critically involved in long-term potentiation (LTP)[13], and synthesis of proteins that maintain such presynaptic mechanisms including synaptophysin and synapsin [20]. However, despite such important role of NMDARs in triggering this cascade, NMDAR antagonists have limited therapeutic potential due to their adverse side-effect profiles. Therefore, alternative treatments for PTSD are needed.

It has been surprisingly discovered herein that PTSD and related diseases are mediated by the PSD95-nNOS protein-protein-interaction (PPI). It has also been surprisingly discovered herein that compounds ultimately modulate the PSD95-nNOS PPI are efficacious in treating post traumatic stress disorder, and related diseases. It is to be understood herein that such compounds may directly or indirectly modulate the PSD95-nNOS PPI. For example, direct modulation illustratively includes those compounds that prevent, decrease, inhibit, or otherwise interfere with the association of PSD95 and nNOS, where that association would contribute at least in part to the disease. Alternatively, indirect modulation illustratively includes those compounds that prevent, decrease, inhibit, or otherwise interfere with the association of PSD95 and nNOS by operating upstream. In one illustrative variation, the indirect modulation is by the administration of an inhibitor of the PPI between NMDA subtype NR2B receptor and PSD95. In another illustrative variation, the indirect modulation is by the administration of an inhibitor or antagonist of the NMDA subtype NR2B receptor.

It has also been surprisingly discovered herein that compounds that selectively antagonize the coupling of PSD95 and nNOS are useful in treating PTSD. In addition, such compounds may circumvent the limitations observed to accompany treatment using NMDAR antagonists. It has also been discovered herein that inhibiting nNOS-PSD95 coupling can block the long-term encoding of conditioned fear even after a fear conditioning session has occurred (i.e., post-trauma). The compounds described herein are useful for ameliorating the long term consequences of trauma. It is appreciated herein that the methods described herein may be most effective when administered shortly after the traumatic event or during trauma recall.

In one illustrative embodiment of the invention, compounds, compositions, unit doses, unit dosage forms, methods, and uses are described herein for treating PTSD and related diseases. In another illustrative embodiment, such compositions, unit doses, unit dosage forms, methods, and uses include a therapeutically effective amount of a modulator of NMDA NR2-PSD95-nNOS signaling. In another embodiment, such compositions, unit doses, unit dosage forms, methods, and uses include a therapeutically effective amount of an inhibitor of PSD95-nNOS protein-protein-interactions (PPIs). In another embodiment, such compositions, unit doses, unit dosage forms, methods, and uses include a therapeutically effective amount of an inhibitor of NMDA NR2-PSD95 PPIs. In another embodiment, such compositions, unit doses, unit dosage forms, methods, and uses include a therapeutically effective amount of a selective antagonist of the NMDA NR2 receptor.

In another embodiment, pharmaceutical compositions containing one or more of the compounds are also described herein. In one aspect, the compositions include a therapeutically effective amount of the one or more compounds for treating a patient with PTSD and/or related diseases. It is to be understood that the compositions may include other component and/or ingredients, including, but not limited to, other therapeutically active compounds, and/or one or more carriers, diluents, excipients, and the like. In another embodiment, methods for using the compounds and pharmaceutical compositions for treating patients with PTSD and/or related diseases are also described herein. In one aspect, the methods include the step of administering one or more of the compounds and/or compositions described herein to a patient with PTSD and/or related diseases. In another aspect, the methods include administering a therapeutically effective amount of the one or more compounds and/or compositions described herein for treating patients with PTSD and/or related diseases. In another embodiment, uses of the compounds and compositions in the manufacture of a medicament for treating patients with PTSD and/or related diseases are also described herein. In one aspect, the medicaments include a therapeutically effective amount of the one or more compounds and/or compositions for treating a patient with PTSD and/or related diseases.

It is appreciated herein that the compounds described herein may be used alone or in combination with other compounds useful for treating PTSD and/or related diseases, including those compounds that may be therapeutically effective by the same or different modes of action. In addition, it is appreciated herein that the compounds described herein may be used in combination with other compounds that are administered to treat other symptoms of PTSD and/or related diseases.

### BRIEF DESCRIPTION OF THE DRAWINGS

FIGS. 1 and 2 show the effects of pre-treating rats with either IC87201 (FIG. 1) or ZL006 (FIG. 2) on percent time freezing following fear conditioning. Acquisition of fear conditioning; test day 1; extinction on day 2; and recall on day 3. Both IC87201 (drug×time interaction F_{(8,84)=}1.5, p=0.001; n=6) and ZL006 (drug×time interaction F_{(8,80)=}3.1, p=0.005; n=6) reduced the percent time freezing on test day 1 (* indicates statistical significance between vehicle and drug pretreated rats Tukey's HSD posthoc test protected by an gas or drug×time ANOVA effect p<0.05). Neither IC8701 nor ZL006 altered the percent time freezing during acquisition (IC87201: drug×time interaction F_{(8,84)=}1.5, p=0.155; ZL006: drug×time interaction F_{(8,80)=}0.7, p=0.673), extinction (IC87201: drug×time interaction F_{(8,84)=}0.7, p=0.899; ZL006: drug×time interaction F_{(8,80)=}0.9, p=0.651), or recall (data not shown). Post conditioning with the compounds described herein does not interfere with consolidation of extinction

FIG. 3 shows the effect of i.p. injection of ZL006 (10 mg/kg, solid triangles), compared to vehicle (solid circles) and a negative control (10 mg/kg, open triangles) on freezing when administered after the acquisition day, and 1 hour prior to the consolidation day. ZL006 showed improved extinction (F(2,14)=10.4, p=0.002; * represents significance between subjects with a Dunnett's posthoc text, p<0.05). ZL006 also showed improved recall (F(2, 14)=4.4, p=0.033, data not shown).

FIG. 4 In vitro binding assay. IC87201 dose-dependently inhibited the interaction between nNOS (1-299) and PSD95 in a plate-binding assay, where nNOS was coated on a 96-well plate and biotinylated PSD95 was added as a ligand. IC50 for IC87201 is 31 mM (n=5) and for tat-nNOS is 0.3 mM (representative of eight experiments). IC87201 reportedly does not disrupt PSD95-cypin (cytosolic interactor) protein-protein interaction in plate binding assay (Florio 2009).

FIG. 5 shows the coimmunoprecipitation experiments showing the effect of ZL006 on nNOS-PSD-95 interaction.

FIG. 6 shows IC87201 attenuates the NMDA-induced increase of cGMP in primary cultured hippocampal neurons dose-dependently attenuated NMDA-induced increases in cGMP, an indirect measurement of nitric oxide production. The IC50 value for IC87201 (n=14-20) is 2.7 mM. cGMP, 3′,5′-cyclic guanosine monophosphate; NMDA, N-methyl-D-aspartic acid; nNOS, neuronal nitric oxide synthase. Disruption of downstream signaling as a result of nNOS-PSD95 inhibition: IC87201 disrupts NMDA receptor induced increase in NO-dependent cGMP production with no effect on cGMP production induced by a NO donor (Florio 2009).

FIG. 7 shows that ZL006 prevents NMDAR-dependent excitotoxicity and cerebral ischemia. Lactate dehydrogenase release from cultured neurons exposed to 50 μM glutamate with 10 μM glycine for 30 min. Morphological changes of neurons (left) and summarized data (right, n=3). Scale bar=60 μm. Values are means±s.e.m., *P<0.05, **P<0.01, ***P<0.001 versus control and versus sham; #P<0.05, ##P<0.01, ###P<0.001 versus vehicle.

FIG. 8 shows concentrations of ZL006 in serum, CSF and brain tissue. ZL006 (1.5 mg kg₋₁) was administered intravenously. Concentrations of ZL006 in serum, brain tissue and CSF were measured at 15 and 60 min after the dosing (μg/ml for serum and CSF, μg/g for brain tissue). Values are means±s.e.m., n=6. *P<0.05.

### DETAILED DESCRIPTION

Several illustrative embodiments of the invention are described by way of the following enumerated clauses:

1. A method for treating PTSD or a related disease in a host animal, the method comprising the step of administering to the host animal a therapeutically effective amount of a composition comprising one or more modulators of NMDA-PSD95-nNOS signaling.

2. The method of clause 1 wherein the host animal is a human.

3. The method of clause 1 or 2 wherein at least one modulator is capable of crossing the blood-brain-barrier.

4. The method of any of the preceding clauses wherein at least one modulator inhibits NMDA signal transduction to PSD95.

5. The method of any of the preceding clauses wherein at least one modulator is an NMDA NR2B receptor antagonist.

6. The method of any of the preceding clauses wherein at least one modulator is compound of formula

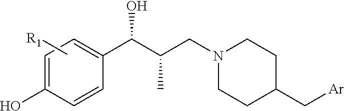

or a pharmaceutically acceptable salt thereof, wherein

Ar is optionally substituted aryl or heteroaryl; and

R₁ represents from 0 to 2 substituents independently selected from the group consisting of amino, hydroxyl, halo, thiol, alkyl, haloalkyl, heteroalkyl, nitro, sulfonic acids and derivatives thereof, and carboxylic acids and derivatives thereof.

7. The method of any of the preceding clauses wherein Ar is optionally substituted phenyl.

8. The method of any of the preceding clauses wherein Ar is phenyl.

9. The method of any of the preceding clauses wherein R₁ represents 0 substituents.

10. The method of any of the preceding clauses wherein the modulator is compound Ro25-6981.

11. The method of any of the preceding clauses wherein the modulator inhibits NMDA-PSD95 protein-protein interactions.

12. The method of any of the preceding clauses wherein the modulator is Tat-NR2B9c.

13. The method of any of the preceding clauses wherein the modulator inhibits PSD95-nNOS protein-protein interactions.

14. The method of any of the preceding clauses wherein at least one modulator is a compound of the formula

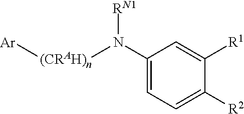

or a pharmaceutically acceptable salt thereof, wherein:

one of R¹ and R² is carboxylic acid or a derivative thereof, and the other is hydroxy or a derivative thereof.

Ar is optionally substituted aryl or optionally substituted heteroaryl;

R^{A} is independently selected in each instance H or alkyl;

R^{N1} is H, acyl, or a nitrogen prodrug forming group, or alkyl, cycloalkyl, heteroalkyl, cycloheteroalkyl, arylalkyl, or heteroarylalkyl, each of which is optionally substituted; and

n is an integer from 1 to about 4.

15. The method of any of the preceding clauses wherein one of R¹ and R² is carboxylic acid or an ester derivative thereof, and the other is hydroxy or an ether derivative thereof.

16. The method of any of the preceding clauses wherein one of R¹ and R² is carboxylic acid or a methyl ester derivative thereof, and the other is hydroxy or a methyl ether derivative thereof.

17. The method of any of the preceding clauses wherein one of R¹ and R² is carboxylic acid, and the other is hydroxy.

18. The method of any of the preceding clauses wherein R¹ is OH or OMe.

19. The method of any of the preceding clauses wherein R¹ is OH.

20. The method of any of the preceding clauses wherein R² is CO₂H or CO₂Me.

21. The method of any of the preceding clauses wherein R² is CO₂H.

22. The method of any of the preceding clauses wherein at least one modulator is a compound of the formula

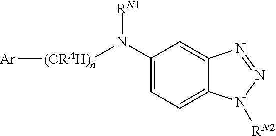

or a pharmaceutically acceptable salt thereof, wherein:

Ar is optionally substituted aryl or optionally substituted heteroaryl;

R^{A} is independently selected in each instance H or alkyl;

R^{N1} and R^{N2} are each independently selected in each instance from the group consisting of H, acyl, and nitrogen prodrug forming groups, and alkyl, cycloalkyl, heteroalkyl, cycloheteroalkyl, arylalkyl, and heteroarylalkyl, each of which is optionally substituted; and

n is an integer from 1 to about 4.

23. The method of any of the preceding clauses wherein Ar is optionally substituted aryl.

24. The method of any of the preceding clauses wherein Ar is substituted aryl.

25. The method of any of the preceding clauses wherein Ar is

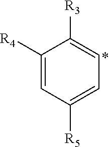

where * indicates the point of attachment;

R³ is hydrogen, hydroxy, or methoxy; and

R⁴ and R⁵ are independently selected from the group consisting of hydrogen, fluoro, chloro, or bromo.

26. The method of any of the preceding clauses wherein Ar is aryl substituted with hydroxy, alkoxy, or a combination thereof.

27. The method of any of the preceding clauses wherein Ar is aryl substituted with halo.

28. The method of any of the preceding clauses wherein Ar is aryl substituted with hydroxy.

29. The method of any of the preceding clauses wherein Ar is aryl substituted with methoxy.

30. The method of any of the preceding clauses wherein Ar is aryl substituted with fluoro.

31. The method of any of the preceding clauses wherein Ar is aryl substituted with chloro.

32. The method of any of the preceding clauses wherein Ar is aryl substituted with bromo.

33. The method of any of the preceding clauses wherein n is an integer from 1 to about 3.

34. The method of any of the preceding clauses wherein n is 3.

35. The method of any of the preceding clauses wherein n is 2.

36. The method of any of the preceding clauses wherein n is 1.

37. The method of any of the preceding clauses wherein R^{N2} is H.

38. The method of any of the preceding clauses wherein R^{N1} is H.

39. The method of any of the preceding clauses wherein R^{N1} is alkyl.

40. The method of any of the preceding clauses wherein R^{N1} is methyl.

41. The method of any of the preceding clauses wherein each R^{A} is H.

42. The method of any of the preceding clauses wherein each R^{A} is methyl.

43. The method of any of the preceding clauses wherein one R^{A} is methyl, and the remaining R^{A} are each H.

44. The method of any of the preceding clauses wherein at least one modulator is a compound of the formula

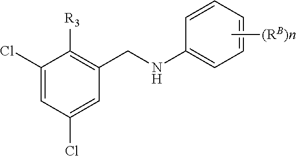

or a pharmaceutically acceptable salt thereof, wherein

R^{B}, independently, is selected from the group consisting of C₁₋₄alkyl, halo, CF₃, OCF₃, C(═O)R^{a}, C(═O)OR^{a}, N(R^{a})₂, C(═O)N(R^{a})₂, NR^{a}C(═O)N(R^{a})₂, OR^{a}, SR^{a}, NO₂, CN, SO₂N(R^{a})₂, SOR^{a}, SO₂R^{a}, and OSO₂CF₃; or two R¹ groups can be taken together with the carbon atoms to which they are attached to form an optionally substituted 5- to 7-membered aliphatic or aromatic ring, and optionally containing one to three heteroatoms selected from the group consisting of oxygen, nitrogen, and sulfur

R³is hydrogen or OH;

R^{a}, independently, is selected from the group consisting of hydro, C₁₋₄alkyl, aryl, and heteroaryl; and

n is an integer 0 through 4.

45. The method of any of the preceding clauses wherein two R^{B} groups are taken together to form a 5- or 6-membered heteroaryl group selected from the group consisting of

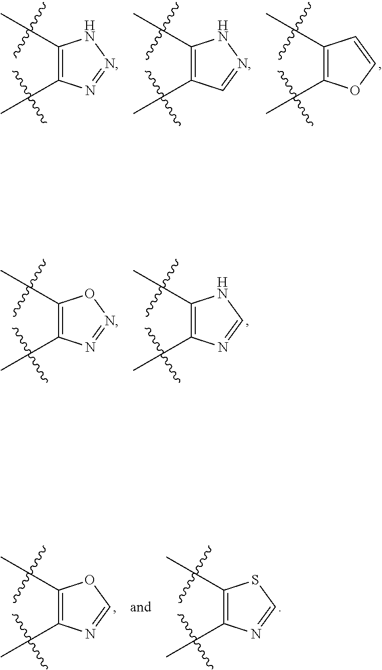

46. The method of any of the preceding clauses wherein two R^{B} groups are taken together, with the phenyl ring to which they are attached, to form a bicyclic aromatic ring system selected from the group consisting of, naphthalene, indene, benzoxazole, benzothiazole, benzisoxazole, benzimidazole, quinoline, indole, benzothiophene, and benzofuran, or two R¹ groups are taken together to form

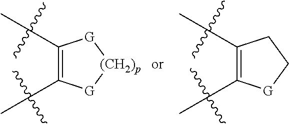

where p is 1 or 2: and G, independently, is C(R^{a})₂, O, S, or NR^{a}.

47. The method of any of the preceding clauses wherein G is independently selected in each instance from C(R^{a})₂ or O.

48. The method of any of the preceding clauses wherein two R^{B} groups are taken together to form

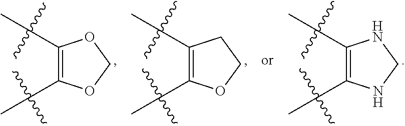 [SMILES: *C1=C(*)OCO1 ; *C1=C(*)OCC1 ; *C1=C(*)NCN1]

49. The method of any of the preceding clauses wherein two R^{B} groups are taken together to form an optionally substituted 5- or 6-membered heteroaryl group selected from the group consisting of

50. The method of any of the preceding clauses wherein at least one modulator is a tetrapeptide or a pentapeptide of the formula

A-B-C-D-E

or a pharmaceutically acceptable salt thereof;

wherein A is absent, or A is Pro or Val; B is Glu, Gln, or Arg; C is Thr; D is Asp, Asn, or His; and E is Val, Leu, or Ile; or

wherein B is Asp when D is Glu; and

where the terminal NH₂ is optionally acylated, such as acetylated, or optionally linked to Tat.

51. The method of any of the preceding clauses wherein at least one modulator is a peptide selected from the group consisting of RQIKIWFQNRRMKWKKNAKAVETDV (SEQ. ID. NO. 1), RQIKIWFQNRRMKWKKAVEATA (SEQ. ID. NO. 2), KNAKAVEDTA (SEQ. ID. NO. 3), KAVEDTA (SEQ. ID. NO. 4), NAKAVETDV (SEQ. ID. NO. 5), VETDV (SEQ. ID. NO. 6), VEDTV (SEQ. ID. NO. 7), VETDV-amide (SEQ. ID. NO. 8), acetyl-VETDV (SEQ. ID. NO. 9), Tat-VETDV (SEQ. ID. NO. 10), PETDV (SEQ. ID. NO. 11), VQTDV (SEQ. ID. NO. 12), VDTDV (SEQ. ID. NO. 13), VRTDV (SEQ. ID. NO. 14), VKTDV (SEQ. ID. NO. 15), VEVDV (SEQ. ID. NO. 16), VESDV (SEQ. ID. NO. 17), VETNV (SEQ. ID. NO. 18), VQTNV (SEQ. ID. NO. 19), VETLV (SEQ. ID. NO. 20), VETEV (SEQ. ID. NO. 21), VDTEV (SEQ. ID. NO. 22), VETHV (SEQ. ID. NO. 23), VETDL (SEQ. ID. NO. 24), VETDI (SEQ. ID. NO. 25), VETDG (SEQ. ID. NO. 26), VETDA (SEQ. ID. NO. 27), and ETDV (SEQ. ID. NO. 28).

52. The method of any of the preceding clauses wherein at least one modulator comprises a catalytically inactive nNOS containing PSD95 binding region.

53. The method of any of the preceding clauses wherein at least one modulator is a catalytically inactive nNOS containing PSD95 binding region.

54. The method of any of the preceding clauses wherein at least one modulator comprises a catalytically inactive nNOS containing residues 1-299 of nNOS.

55. The method of any of the preceding clauses wherein at least one modulator is a catalytically inactive nNOS containing residues 1-299 of nNOS.

56. The method of any of the preceding clauses wherein at least one modulator is a Tat-nNOS (1-299) fusion protein, where the Tat is derived from HIV protein.

57. The method of any of the preceding clauses wherein at least one modulator is a fusion protein that comprises Tat-nNOS (16-130), it being understood that the fusion protein may optionally further comprise an additional N-terminal sequence, an additional C-terminal sequence, or both.

58. The method of any of the preceding clauses wherein at least one modulator is a fusion protein that comprises a mutant of Tat-nNOS (16-130), it being understood that the fusion protein may optionally further comprise an additional N-terminal sequence, an additional C-terminal sequence, or both, and that the mutant includes at least the corresponding residues E108, T109, T110, and F111.

59. The method of any of the preceding clauses wherein at least one modulator is a LV-nNOS (1-133)-GFP fusion protein.

60. The method of any of the preceding clauses wherein Tat is YGRKKRRQRRR (SEQ. ID. NO. 29).

61. The method of any of the preceding clauses wherein at least one modulator does not inhibit the syntropin-nNOS protein-protein interaction.

62. The method of any of the preceding clauses wherein at least one modulator does not inhibit the cypin-nNOS protein-protein interaction.

63. The method of any of the preceding clauses wherein at least one modulator does not inhibit the Capon-nNOS protein-protein interaction.

64. The method of any of the preceding clauses wherein the modulator does not inhibit the PSD95-SynGAP protein-protein interaction.

65. The method of any of the preceding clauses wherein at least one modulator does not inhibit NMDA receptor conductance.

66. The method of any of the preceding clauses wherein the modulator does not inhibit nNOS catalytic activity.

67. The method of any of the preceding clauses wherein at least one modulator inhibits NMDA receptor induced increase in NO-dependent cGMP production.

68. The method of any of the preceding clauses wherein at least one modulator does not substantially affect cGMP production induced by a NO donor.

69. The method of any of the preceding clauses wherein at least one modulator does not substantially affect memory in the patient.

70. The method of any of the preceding clauses wherein at least one modulator does not substantially affect spatial memory in the patient.

71. The method of any of the preceding clauses wherein at least one modulator does not substantially affect working memory in the patient.

72. The method of any of the preceding clauses wherein at least one modulator does not substantially affect learning in the patient.

In another embodiment, at least one modulator is

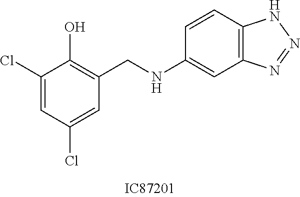 [SMILES: Oc1c(Cl)cc(Cl)cc1CNc1ccc2[nH]nnc2c1]

or a pharmaceutically acceptable salt thereof.

In another embodiment, at least one modulator is

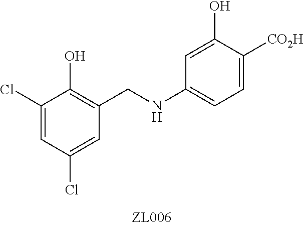 [SMILES: O=C(O)c1ccc(NCc2cc(Cl)cc(Cl)c2O)cc1O]

or a pharmaceutically acceptable salt thereof.

In another embodiment, at least one modulator is a compound selected from

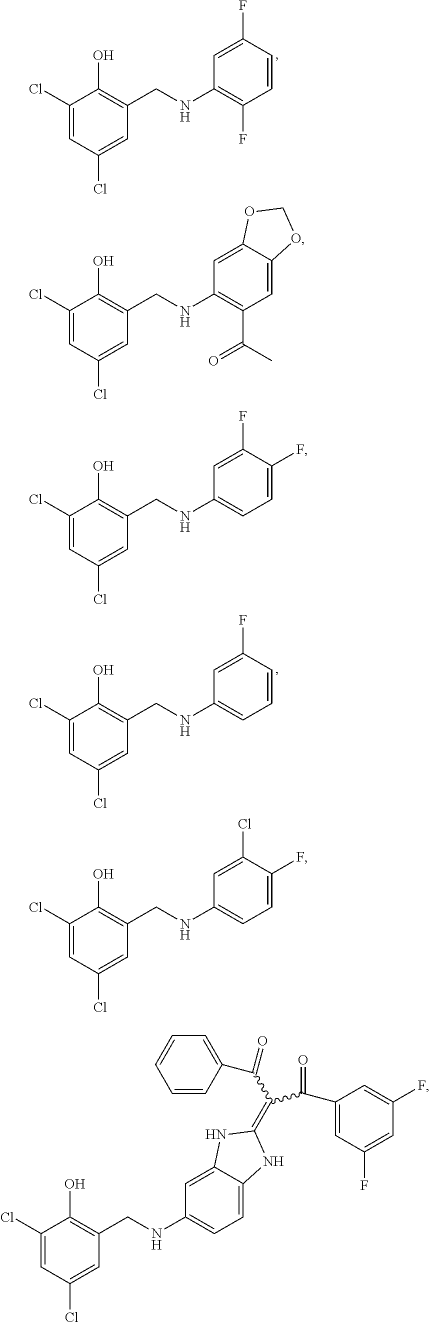

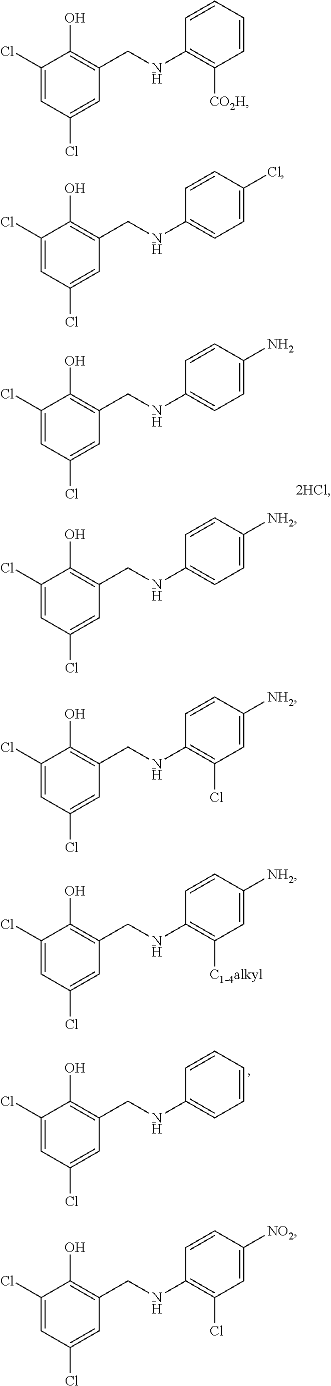

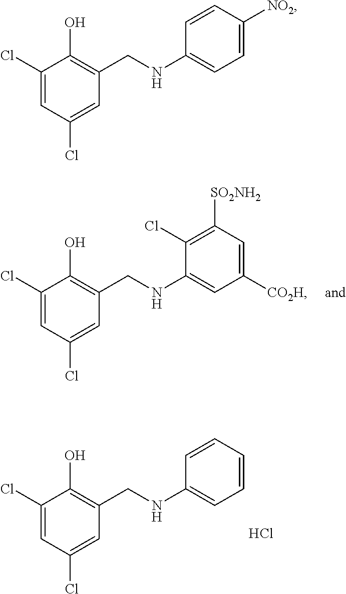 [SMILES: O=[N+]([O-])c1ccc(NCc2cc(Cl)cc(Cl)c2O)cc1 ; NS(=O)(=O)c1cc(C(=O)O)cc(NCc2cc(Cl)cc(Cl)c2O)c1Cl ; Oc1c(Cl)cc(Cl)cc1CNc1ccccc1 ; Cl]

or a pharmaceutically acceptable salt of any of the foregoing, and combinations thereof. Additional details regarding the preparation of the foregoing examples are described in WO 2005/097090, the disclosure of which is incorporated herein by reference.

In each of the foregoing and following embodiments, it is to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non crystalline and/or amorphous forms of the compounds.

It is to be understood that such derivatives may include prodrugs of the compounds described herein, compounds described herein that include one or more protection or protecting groups, including compounds that are used in the preparation of other compounds described herein.

It is appreciated herein that the compounds advantageously do not inhibit other protein-protein interactions.

It is appreciated herein that the compounds advantageously do not have any effect on NOS catalytic activity.

It is appreciated herein that the compounds advantageously do not have any effect on motor activity.

It is appreciated herein that the compounds advantageously do not have any effect on normal nociception.

Without being bound by theory, it is believed herein that fusion proteins that include Tat and related sequences are capable of crossing the BBB.

In another embodiment, the compositions, unit doses, unit dosage forms, methods, and uses are described herein for treating PTSD that has been diagnosed in a patient.

The compounds described herein may contain one or more chiral centers, or may otherwise be capable of existing as multiple stereoisomers. It is to be understood that in one embodiment, the invention described herein is not limited to any particular sterochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may be include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

The family of glutamate receptors is divided into ionotropic sub-types comprising α-amino-3-hydroxy-5-methyl-4-isoxazole proprionic acid (AMPA), kainate and NMDA receptors and metabotropic subtypes mGluR1-8 based on sequence homology, pharmacology, and electrophysiological properties. The NMDA receptor is widely distributed in mammalian brain. It has been reported that activation of the NMDA receptor leads to Ca²⁺ influx as well as regulation of other signaling pathways including neuronal nitric oxide synthase (nNOS, also called NOS-1). The activation of nNOS via NMDA receptors requires interaction with the scaffold protein PSD-95 (postsynaptic density 95 kDa), which forms an NMDAR/PSD-95/nNOS complex.

NMDA receptors are tetrameric and typically contain two NR1 and two NR2 subunits (also called GluN1 and GluN2). Opening of the NMDA receptor channel requires the binding of both glutamate on the NR2 subunit and the co-agonist glycine on NR1. Additional binding sites include a site within the channel at which use-dependent antagonists such as ketamine and MK-801 can bind. NMDA receptors can also contain NR3 subunits that modulate receptor properties by, for example, reducing both whole-cell currents and single-channel conductance. It has been reported that at a neuronal resting membrane potential of about −65 mV, NMDA receptors undergo channel block by extracellular Mg²⁺. The AMPA receptor-induced depolarisation of neurons allows lifting of the voltage-dependent block by Mg²⁺, via influx of mainly Ca²⁺ and to a lesser extent Na⁺. The Ca²⁺ influx triggers a variety of intracellular signalling cascades including activation of a Ca²⁺/calmodulin complex, which in turn stimulates nNOS leading to the production of nitric oxide. It has been reported that over-activation of the NMDA receptor may cause excitotoxicity due to excessive Ca²⁺ influx. However, other reported studies show that Ca²⁺ blockers alone are not protective against this process, suggesting that activation of additional intracellular cascades coupled to the NMDA receptor, such as nitric oxide, are also important in excitotoxicity.

The postsynaptic density (PSD) is a membrane-associated megaorganelle specialized for postsynaptic signal transduction and processing. The PSD is located at the head of dendritic spines and is a disc-like structure about 200-800 nm wide and 30-50 nm thick occupying about 10% of the surface area of the spine. It has been reported that at the PSD, synaptic-expressed proteins are aligned with the presynaptic active zone. There are four PSD family members classified according to their molecular weight, including, PSD-95 (SAP-90), PSD-93 (Chapsyn-110), and synaptic associated proteins 97 kDa and 102 kDa (SAP-97 and SAP-102, respectively). Briefly, SAP-97 is found in the pre- and postsynaptic compartments, whereas PSD-95, PSD-93 and SAP-102 are found at the postsynaptic membrane of excitatory synapses. The postsynaptic-expressed PSD proteins are located close to the membrane at a mean distance of 12 nm, where PSD-95 and PSD-93 form multimers mediated by N-terminal “head to head” interactions.

Post-synaptic density protein 95 (PSD-95) uses a combination of three PSD-95/Drosophila disc large/ZO-1 homology (PDZ) domains to recruit proteins, including nNOS to the NMDA receptor. This close positioning of nNOS to the NMDA receptor allows for the effective activation of nNOS by calcium entering through the receptor. It has also been reported that overactivation of the NMDA receptor results in high levels of NO that may be toxic. Of the three NOS isoforms, nNOS is unique in that it contains an N-terminal PSD95-binding domain, which is required for functional coupling of nNOS to the NMDA receptor-PSD95 complex. It has been reported that suppression of PSD95 expression with antisense oligonucleotides decreased NMDA-induced NO production and NMDA mediated excitoxicity in cultured neurons without affecting NMDA receptor channel properties. Similar results were obtained with interfering RNA.

The family of PSD proteins is made of three PDZ (PSD-95/DlgA/Zo-1) domains, a SH3 (Src homology 3) domain and a GK (guanylate kinase) domain, which has lost its catalytic activity. PSD-95 interacts with both ionotropic and metabotropic glutamate receptors via protein-protein interactions and plays a role in their precise assembly and spatial organization as well as coupling these receptors to downstream signaling events.

Nitric oxide synthases are divided into three major isoforms: neuronal (nNOS or NOS-1), inducible (iNOS or NOS-2) and endothelial (eNOS or NOS-3). The human neuronal isoform (nNOS) is a 1434 amino acid protein of 160.8 kDa. The gene encoding nNOS is located on chromosome 12 (12q24.2-12q24.3), incorporates 29 exons and shows sequence conservation through many species. nNOS has been identified in developing and mature neurons, but is also present in skin and bronchial epithelium. In skeletal muscles, nNOS binds to α₁-syntrophin and caveolin-3 to form a complex with sarcolemmal dystrophin.

It is reported that PSD-95 is a scaffolding protein that binds both NMDARs and nNOS at excitatory synapses and assembles them into a macromolecular signaling complex. Activation of nNOS depends on its association with PSD-95 and on NMDAR-mediated calcium influx. The brain nNOS exists in particulate and soluble forms and is distributed mainly in the cytosol. nNOS is targeted to membranes by binding to syntrophin, PSD-95, PSD-93 or synapse-associated protein-90. It is also reported that neurons lacking PSD-95 or nNOS show reduced excitotoxic vulnerability.

One consequence of NMDA receptor activation is the entry of calcium, which binds to calmodulin to activate downstream effectors including neuronal nitric oxide synthase (nNOS). The subsequent overproduction of nitric oxide (NO) is thought to promote hyperalgesia. At physiological levels, NO plays a key role as a second messenger. However, it is reported that at elevated levels, the toxicity of NO dominates. Because of its non-enzymatic degradation and brief half-life, NO levels are primarily regulated by its synthetic enzyme, NOS.

As used herein, the term “alkyl” includes a chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. It is to be further understood that in certain embodiments alkenyl and/or alkynyl may each be advantageously of limited length, including C₂-C₂₄, C₂-C₁₂, C₂-C₈, C₂-C₆, and C₂-C₄. It is appreciated herein that shorter alkyl, alkenyl, and/or alkynyl groups may add less lipophilicity to the compound and accordingly will have different pharmacokinetic behavior. Illustrative alkyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, amino alkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylic acid and derivatives thereof” includes the group CO₂H and salts thereof, and esters and amides thereof, and CN.

As used herein, the term “sulfinic acid or a derivative thereof” includes SO₂H and salts thereof, and esters and amides thereof.

As used herein, the term “sulfonic acid or a derivative thereof” includes SO₃H and salts thereof, and esters and amides thereof.

As used herein, the term “sulfonyl” includes alkylsulfonyl, alkenylsulfonyl, alkynylsulfonyl, heteroalkylsulfonyl, heteroalkenylsulfonyl, heteroalkynylsulfonyl, cycloalkylsulfonyl, cycloalkenylsulfonyl, cycloheteroalkylsulfonyl, cycloheteroalkenylsulfonyl, arylsulfonyl, arylalkylsulfonyl, arylalkenylsulfonyl, arylalkynylsulfonyl, heteroarylsulfonyl, heteroarylalkylsulfonyl, heteroarylalkenylsulfonyl, heteroarylalkynylsulfonyl, acylsulfonyl, and the like, each of which is optionally substituted.

As used herein, the term “phosphinic acid or a derivative thereof” includes P(R)0₂H and salts thereof, and esters and amides thereof, where R is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted.

As used herein, the term “phosphonic acid or a derivative thereof” includes PO₃H₂ and salts thereof, and esters and amides thereof.

As used herein, the term “hydroxylamino and derivatives thereof” includes NHOH, and alkyloxylNH alkenyloxylNH alkynyloxylNH heteroalkyloxylNH heteroalkenyloxylNH heteroalkynyloxylNH cycloalkyloxylNH cycloalkenyloxylNH cycloheteroalkyloxylNH cycloheteroalkenyloxylNH aryloxylNH arylalkyloxylNH arylalkenyloxylNH arylalkynyloxylNH heteroaryloxylNH heteroarylalkyloxylNH heteroarylalkenyloxylNH heteroarylalkynyloxylNH acyloxy, and the like, each of which is optionally substituted.

As used herein, the term “hydrazino and derivatives thereof” includes alkylNHNH, alkenylNHNH, alkynylNHNH, heteroalkylNHNH, heteroalkenylNHNH, heteroalkynylNHNH, cycloalkylNHNH, cycloalkenylNHNH, cycloheteroalkylNHNH, cycloheteroalkenylNHNH, arylNHNH, arylalkylNHNH, arylalkenylNHNH, arylalkynylNHNH, heteroarylNHNH, heteroarylalkylNHNH, heteroarylalkenylNHNH, heteroarylalkynylNHNH, acylNHNH, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, amino alkyl, C₁-C₆ alkylaminoalkyl, (C₁-C₆ alkyl)(C₁-C₆ alkyl)aminoalkyl, alkylcarbonylamino alkyl, N—(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165; and Pathalk et al., Enzymic protecting group techniques in organic synthesis, Stereosel. Biocatal. 775-797 (2000). It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Illustrative esters, also referred to as active esters, include but are not limited to 1-indanyl, N-oxysuccinimide; acyloxyalkyl groups such as acetoxymethyl, pivaloyloxymethyl, β-acetoxyethyl, β-pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, a-ethoxycarbonyloxyethyl, β-ethoxycarbonyloxyethyl, and the like; dialkylaminoalkyl groups, including di-lower alkylamino alkyl groups, such as dimethylaminomethyl, dimethylaminoethyl, diethylaminomethyl, diethylamino ethyl, and the like; 2-(alkoxycarbonyl)-2-alkenyl groups such as 2-(isobutoxycarbonyl)pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl and optionally substituted heteroaryl(C₂-C₁₆)alkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and vehicles.

Illustrative routes of oral administration include tablets, capsules, elixirs, syrups, and the like.

Illustrative routes for parenteral administration include intravenous, intraarterial, intraperitoneal, epidurial, intrathecal, intraurethral, intrasternal, intramuscular and subcutaneous, as well as any other art recognized route of parenteral administration. Illustratively, compounds may be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

Depending upon the disease as described herein, the route of administration and/or whether the compounds and/or compositions are administered locally or systemically, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the foregoing illustrative dosages and dosing protocols, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

The effective use of the compounds, compositions, and methods described herein for treating or ameliorating one or more effects of a PTSD and related diseases using one or more compounds described herein may be based upon animal models, such as murine, canine, porcine, and non-human primate animal models of disease.

The following publications, and each of the additional publications cited herein are incorporated herein by reference:

1. Liebschutz, J., et al., PTSD in urban primary care: high prevalence and low physician recognition. J Gen Intern Med, 2007. 22(6): p. 719-26.

2. Stein, M. B., et al., Posttraumatic stress disorder in the primary care medical setting. Gen Hosp Psychiatry, 2000. 22(4): p. 261-9.

3. Blake, D. D., J. D. Cook, and T. M. Keane, Posttraumatic-Stress-Disorder and Coping in Veterans Who Are Seeking Medical-Treatment. Journal of Clinical Psychology, 1992. 48(6): p. 695-704.

4. Stein, D. J., J. C. Ipser, and S. Seedat, Pharmacotherapy for post traumatic stress disorder (PTSD). Cochrane Database Syst Rev, 2006(1): p. CD002795.

5. Fani, N., et al., Attention bias toward threat is associated with exaggerated fear expression and impaired extinction in PTSD. Psychol Med, 2012. 42(3): p. 533-43.

6. Debiec, J., D. E. Bush, and J. E. LeDoux, Noradrenergic enhancement of reconsolidation in the amygdala impairs extinction of conditioned fear in rats—a possible mechanism for the persistence of traumatic memories in PTSD. Depress Anxiety, 2011. 28(3): p. 186-93.

7. Itzhak, Y., Role of the NMDA receptor and nitric oxide in memory reconsolidation of cocaine-induced conditioned place preference in mice Ann N Y Acad Sci, 2008. 1139: p. 350-7.

8. Resstel, L. B., F. M. Correa, and F. S. Guimaraes, The expression of contextual fear conditioning involves activation of an NMDA receptor-nitric oxide pathway in the medial prefrontal cortex. Cereb Cortex, 2008. 18(9): p. 2027-35.

9. Kelley, J. B., et al., Impairments in fear conditioning in mice lacking the nNOS gene. Learn Mem, 2009. 16(6): p. 371-8.

10. Kelley, J. B., K. L. Anderson, and Y. Itzhak, Pharmacological modulators of nitric oxide signaling and contextual fear conditioning in mice. Psychopharmacology (Berl), 2010. 210(1): p. 65-74.

11. Kelley, J. B., et al., Long-term memory of visually cued fear conditioning: roles of the neuronal nitric oxide synthase gene and cyclic AMP response element-binding protein. Neuroscience, 2011. 174: p. 91-103.

12. Zoubovsky, S. P., et al., Working memory deficits in neuronal nitric oxide synthase knockout mice: potential impairments in prefrontal cortex mediated cognitive function. Biochem Biophys Res Commun, 2011. 408(4): p. 707-12.

13. Lange, M. D., et al., Heterosynaptic long-term potentiation at interneuron-principal neuron synapses in the amygdala requires nitric oxide signalling. J Physiol, 2012. 590(Pt 1): p. 131-43.

14. Schafe, G. E., et al., Memory consolidation of Pavlovian fear conditioning requires nitric oxide signaling in the lateral amygdala. Eur J Neurosci, 2005. 22(1): p. 201-11.

15. Overeem, K. A. and L. Kokkinidis, Nitric oxide synthesis in the basolateral complex of the amygdala is required for the consolidation and expression of fear potentiated startle but not shock sensitization of the acoustic startle. Neurobiol Learn Mem, 2012. 97(1): p. 97-104.

16. Gallo, E. F. and C. Iadecola, Neuronal nitric oxide contributes to neuroplasticity-associated protein expression through cGMP, protein kinase G, and extracellular signal-regulated kinase. J Neurosci, 2011. 31(19): p. 6947-55.

17. Ota, K. T., et al., Synaptic plasticity and NO-cGMP-PKG signaling regulate pre- and postsynaptic alterations at rat lateral amygdala synapses following fear conditioning. PLoS One, 2010. 5(6): p. e11236.

18. Ota, K. T., et al., Synaptic plasticity and NO-cGMP-PKG signaling coordinately regulate ERK-driven gene expression in the lateral amygdala and in the auditory thalamus following Pavlovian fear conditioning. Learn Mem, 2010. 17(4): p. 221-35.

19. Ota, K. T., et al., The NO-cGMP-PKG signaling pathway regulates synaptic plasticity and fear memory consolidation in the lateral amygdala via activation of ERK/MAP kinase. Learn Mem, 2008. 15(10): p. 792-805.

20. Johansen, J. P., et al., Molecular mechanisms of fear learning and memory. Cell, 2011. 147(3): p. 509-24.

21. Florio et al. British Journal of Pharmacology (2009) 158, 494-506.

22. Zhou et al. Nature medicine, Volume 16 Number 12 December 2010.

The following examples further illustrate specific embodiments of the invention; however, the following illustrative examples should not be interpreted in any way to limit the invention.

### EXAMPLES

Abbreviations: 7-NI, 7-nitroindazole; ANOVA, analysis of variance; BH4, tetrahydrobiopterin; BSA, bovine serum albumin; CCI, chronic constriction injury; cGMP, 3!,5!-cyclic guanosine monophosphate; DMSO, dimethyl sulphoxide; EGTA, ethylene glycol tetraacetic acid; GST, glutathione S-transferase; L-NAME, NG-nitro-L-arginine methyl ester; MED, minimum effective dose; NMDA, N-methyl-D-aspartic acid; NOS, nitric oxide synthase; nNOS, neuronal NOS; PBS, phosphate buffered saline; PDZ, PSD95/Drosophila disc large/ZO-1 homology; PSD95, postsynaptic density protein 95; SNL, spinal nerve ligation.

### EXAMPLE

Post-Traumatic Stress Disorder (PTSD) is an illness precipitated by exposure to traumatic event(s) that arouses life-threatening fear or horror. PTSD has been described as having clusters of symptoms including associative fear memory symptoms, which include long-lasting conditioned fear responses, and non-associative fear symptoms, which include generalized behavioral sensitization to novelty or stress. Depending on the type and severity of the traumatic experience and perceived personal vulnerability, the estimated lifetime prevalence of PTSD among adult Americans is 7.8% with current prevalence of 3.5%. The conditioned fear test is a well established model of the associative fear memories that are the cardinal symptoms of PTSD. The model demonstrates that blocking protein-protein interactions between the NMDA glutamate receptor-postsynaptic density protein 95 (PSD95)-nitric oxide synthase (NOS) system is beneficial for treating PTSD symptoms.

### EXAMPLE

Conditioned Fear Test. The conditioning chamber (Hamilton-Kinder, Paolo Alto, Calif.) is equipped to present light and tone cues along with a video camera set up to record behavior. The entire chamber is enclosed in a sound attenuated box. The animals went through 5 days of experimentation: Day 1 Habituation; Day 2 Conditioning; Day 3 Fear recall testing; Day 4 Extinction Training; and Day 5 Extinction Recall Test. Day 1 Habituation: Rats are exposed to the chambers for 10 min. Day 2 Conditioning: Rats are given five presentations of the tone CS (4 kHz, 80 dB, 20 sec) each co-terminating with a 0.8 mA foot shock lasting 0.5-s (US). The first CS-US pairing is presented 120 s into the session and the inter-trial interval (ITI) between CS-US presentations is 105 s on average (range 90-120). Conditioning sessions last about 11 minutes. Day 3 Fear recall tests: Twenty-four hours following the acquisition training, rats are given five 20-s CS presentations in the absence of the US. The first tone is presented 120 s into the session and the inter-trial interval (ITI) between CS-US presentations is 105 s on average (range 90-120). Percent time spent freezing is measured during each tone as an indication of fear recall. Recall test sessions last about 11 minutes. Extinction Training: Rats are exposed to 20 presentations of the CS in the absence of the US (mean ITI 180 s, range 120 to 240 s). Extinction Recall Test: 10 presentations of the CS only (mean ITI 180 s, range 120 to 240 s) are given 24 hrs after extinction.

### EXAMPLE

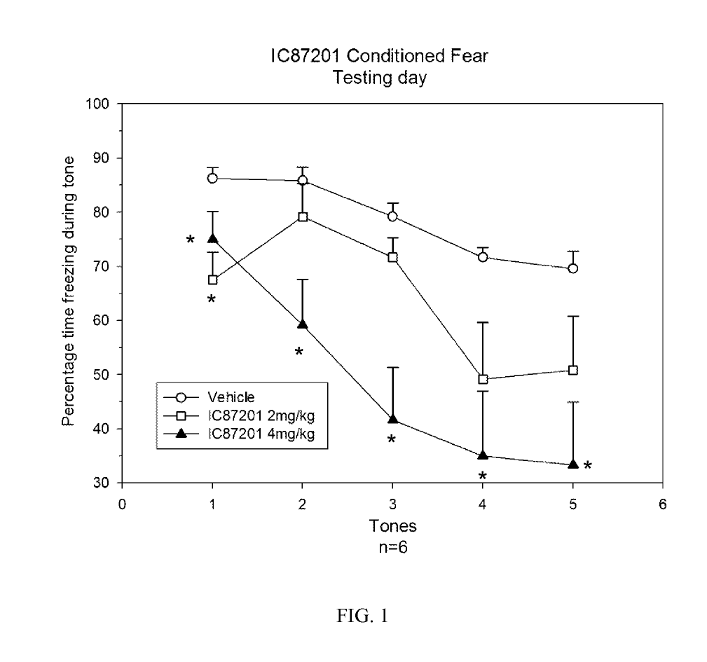

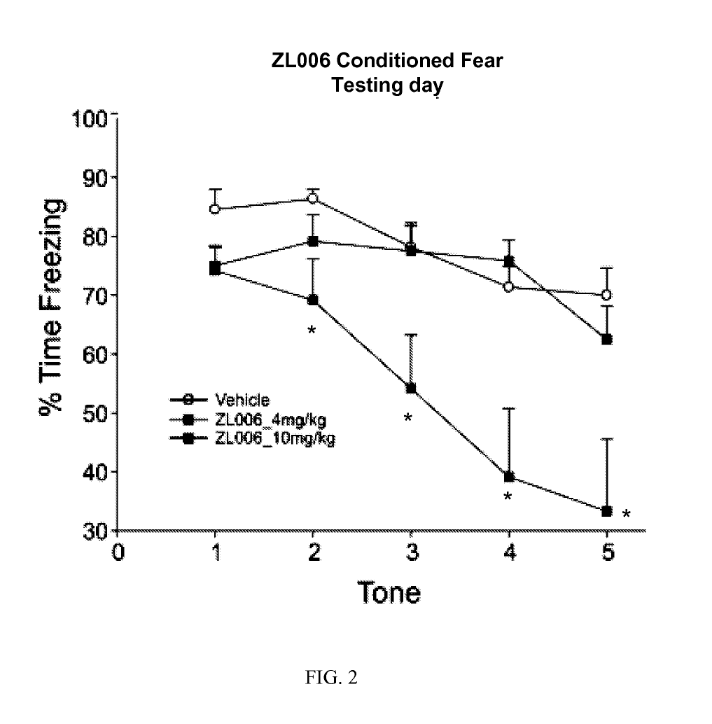

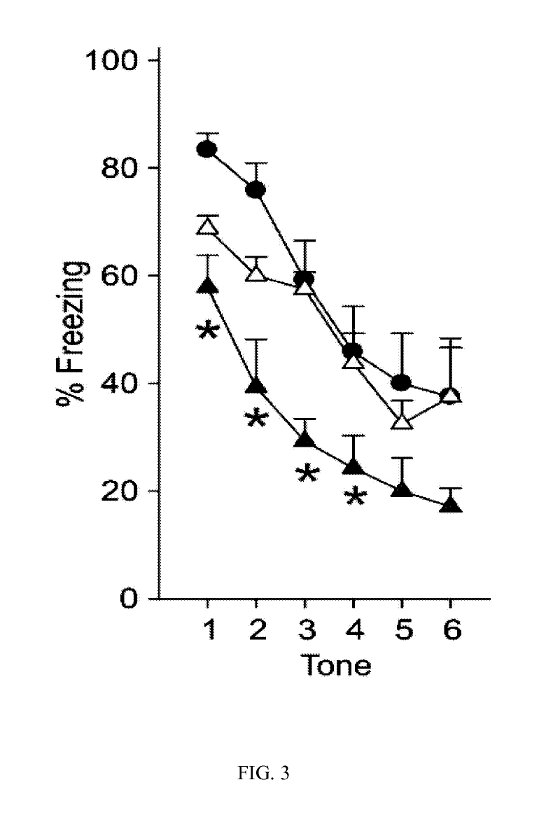

Results. Treatment with two different agents, IC87201 (IC) and ZL006 (ZL), 5-60 min following the day 2 conditioning session (i.e., after the ‘trauma’ had occurred and the associative fear memory formation had begun) resulted in a significant attenuation of conditioned fear response when tested 24 hours later (day 3 fear recall) with both compounds, whereas vehicle treatment (i.e., placebo) showed expression of robust conditioned fear, as shown in FIGS. 1-3.

When compared to their respective vehicle groups, there was identical acquisition of conditioned fear with both the IC and ZL groups, clearly showing that all groups experienced similar ‘trauma’ and ‘conditioning of fear.

Similarly, compared to their respective vehicle groups, both IC and ZL treated groups showed normal patterns of fear extinction, suggesting that treatment with the compounds does not interfere with an individual's ability to erase these fear memories with repeated exposure (e.g., cognitive-behavior therapies that are commonly used along with medications in PTSD subjects).

Compounds described herein do not block appropriate fear responses to actual stimuli. Compounds described herein block development of conditioned fear expression 24 hrs later when given immediately following a fear conditioning session. Compounds described herein administered post conditioning do not interfere with fear extinction.

### EXAMPLE

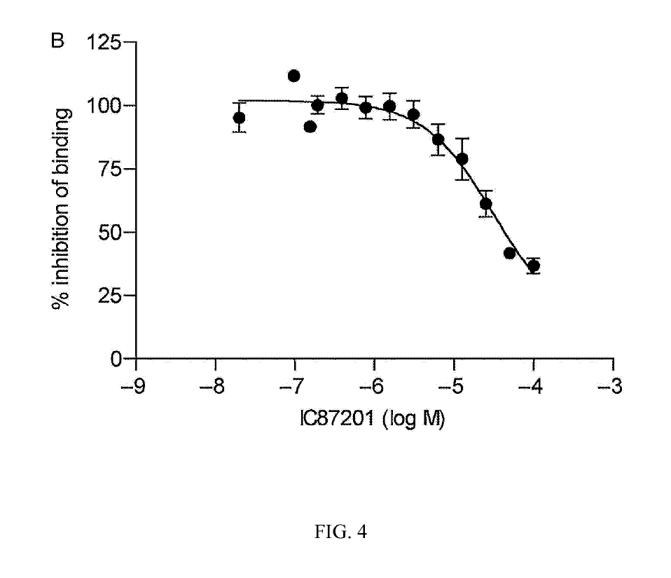

In vitro nNOS-PSD95 interaction assay. Recombinant nNOS (amino acids 1-299, 5 mg·mL-1), cleaved from GST-nNOS, was used to coat wells of an Immulon 96-well plate. After blocking non-specific sites with SEA block (Pierce) and further washing, biotinylated PSD95 (12.5 nM) was added as ‘ligand’ and binding continued for 2 h at room temperature before the reaction was terminated by repeated washing with PBS containing 0.05% Tween 20. The biotinylated PSD95-nNOS complex was detected by streptavidin-europium (Perkin-Elmer, Waltham, Mass., USA). After release of the europium by an enhancement solution (Perkin-Elmer), the increased fluorescence was measured using a DELFIA research fluorimeter (Perkin-Elmer). Using this in vitro binding assay, a high throughput screen was carried out to identify small molecule inhibitors which can disrupt the protein-protein interaction between nNOS and PSD95 Inhibitors were tested for their ability to block the NMDA-induced increase in cGMP production in neuronal cultures (FIG. 4). Without being bound by theory, it is believed herein that the assay is an indirect measurement of NO production in neuronal cultures.

### EXAMPLE

Disurption of nNOS-PSD95 complex in neuronal cultures and in animals: ZL006 inhibits the in co-immunoprecipitation of nNOS-PSD95 complex in neuronal cultures.

### EXAMPLE

Co-immunoprecipitation. Cultured neurons, organotypic hippocampal slice cultures (OHSCs) or the cortices of mice were lysed and centrifuged. The supernatant was preincubated with protein G-Sepharose beads (Sigma-Aldrich) and then centrifuged to obtain the target supernatant. The antibodyconjugated protein G-Sepharose beads were incubated with the target supernatant, centrifuged, washed and heated the beads to elute bound proteins and analyzed proteins by immunoblotting.

### EXAMPLE

Coimmunoprecipitation. Neurons were washed in PBS twice, and then lysed in 400 μl buffer A (50 mM Tris-HCl [pH 7.4], 150 mM NaCl, 1 mM EDTA-Na, 1% NP-40, 0.02% sodium azide, 0.1% SDS, 0.5% sodium deoxycholate, 1% PMSF, 1‰ aprotinin, 1‰ leupeptin, and 0.5‰ pepstatin A). The lysates were centrifugated at 12,000×g for 15 min at 4° C. The OHSCs or cortex of mice was homogenized in ice-cold lysis buffer A. After lysis for 15 min, samples were centrifuged at 20,000×g for 15 min. The supernatant were preincubated for 1 h at 4° C. with 0.025 ml of protein G-sepharose beads (Sigma-Aldrich) and then centrifuged to remove proteins that adhered nonspecifically to the beads and obtain the target supernatant for following IP experiment. Protein G Sepharose beads were incubated with antibodies (rabbit antibody to nNOS, 1:100, Affinity BoReagents; rabbit antibody to PSD95, 1:100, Cell Signaling Technology; and rabbit antibody to NR2B antibody, 1:200, Chemicon) for 3-4 h. The antibodies-conjugated protein G-sepharose beads and the target supernatant were added for incubation overnight. Immune complexes were isolated by centrifugation, washed 4 times with 0.05 M HEPES buffer, pH 7.1, containing 0.15% Triton X-100, 0.15 M NaCl, and 0.1×10⁻³ M sodium orthovanadate, and bound proteins were eluted by heating at 100° C. in loading buffer. Proteins were analyzed by immunoblotting.

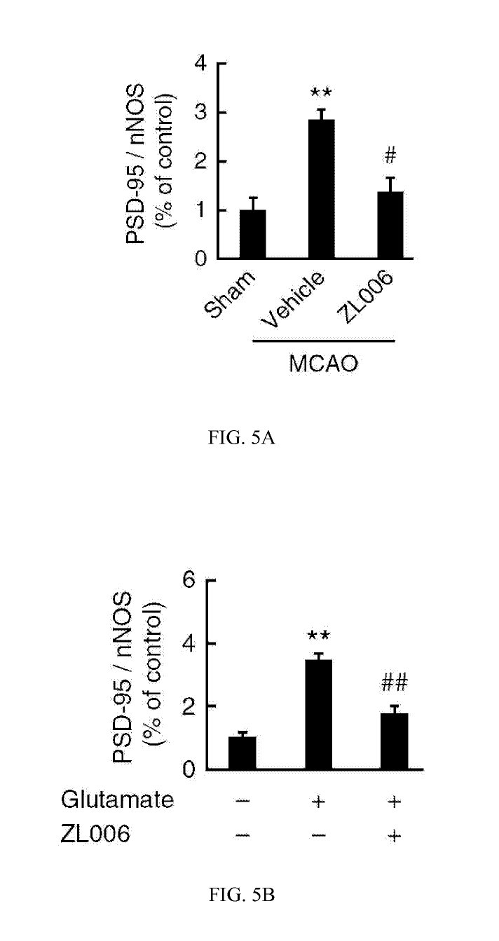

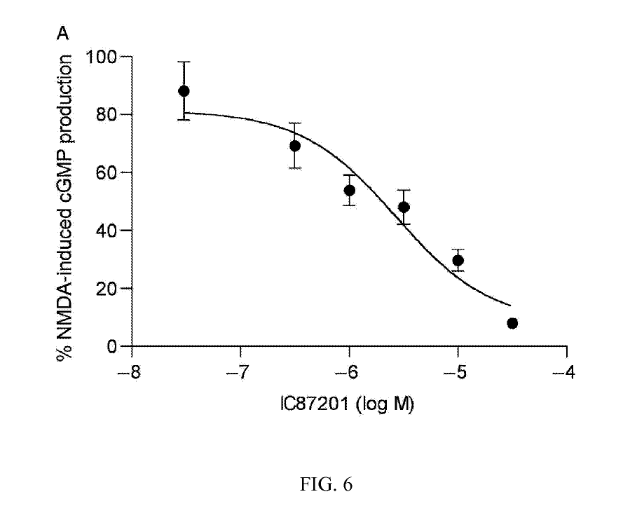

Samples were immunoprecipitated and analyzed by western blotting with the indicated antibodies (IP for nNOS, and WB for PSD-95), and analyzed for (a) nNOS-PSD-95 complex amounts in the cortex of mice subjected to 90 min MCAO and 30 min reperfusion (n=3), as shown in FIG. 5A in wild type mice after MCAO, and (b) nNOS-PSD-95 complex amounts in neurons exposed to 50 μM glutamate with 10 μM glycine for 30 min (n=3). Values are means±s.e.m., **P<0.01 versus sham in c and versus control in d; #P<0.05, ##P<0.01 versus vehicle in FIG. 5A and versus glutamate in FIG. 5B. Coimmunoprecipitation experiments did not show a significant effects of ZL006 on nNOS-PSD95 interaction in the non-ischemic cortex (n=3, P=0.200, ZL006 vs vehicle).

ZL006 blocks the ischemic induced increase in nNOS-PSD95 interaction in wild type mice with focal cerebral ischemic damage after middle cerebral artery occlusion (MCAO) and reperfusion but not in nNOS−/−mice.

### EXAMPLE

ZL006 does not inhibit the co-immunoprecipitation of nNOS-Capon (carboxy-terminal PDX ligand of nNOS) or PSD95-SynGAP (synaptic GTPase activation protein) interaction in ischemic cortexes. ZL006 is specific for nNOS-PSD95 interaction and does not show an effect on (a) nNOS-CAPON (carboxy-terminal PDZ ligand of nNOS) interaction in the ischemic cortexes (P=0.5955, n=3, ZL006 vs vehicle) or (b) PSD95-SynGAP (synaptic GTPase activating protein) interaction in the ischemic cortexes (P=0.6250, n=3, ZL006 vs vehicle). Mice were subjected to 90 min MCAO and 30 min reperfusion and treated with ZL006 (1.5 mg kg-1, i.v.) 15 min before MCAO.

### EXAMPLE

NOS activity assay. Nitric oxide synthase activities in the hippocampus were measured using a conventional assay. Briefly, the hippocampus was homogenized in ice-cold PBS, pH 7.4, and centrifuged at 10,000 g for 20 min at 4° C. The supernatant was ultracentrifuged at 100,000×g for 15 min at 4° C. using a 300 kDa molecular weight cut-off filter by centrifugation. NOS activity in the filtrates was measured using a commercially available kit (Calbiochem). To measure nNOS activity, 1 mM L-nomega-iminoethyl-L-ornithine (Sigma-Aldrich), a selective eNOS inhibitor, was added into the reaction mixture. Neuronal NOS activity was computed by subtracting the iNOS activity from the total NOS activity with the inhibited fraction of eNOS. Inducible NOS activity was measured by adding EGTA at 3 mM to chelate free Ca²⁺ from the reaction mixture. NOS activities were expressed as unit (U). One U was defined as nanomoles of NO formed in 1 min by 1 mg of protein. ZL006 does not inhibit NOS activity. The homogenates from the cortex of mice were treated with 10 μM ZL006 or 1.0 mM viny-L-NIO for 30 min, and then NOS activities were measured. ZL006 does not inhibit NOS activity or NOS catalytic activity (total NOS activity (n=3, *P<0.05, Viny-L-NIO vs vehicle); nNOS activities (n=3, **P<0.01, Viny-L-NIO vs vehicle)).

### EXAMPLE

Electrophysiological recordings. Hippocampal slices were prepared from male SD rats as described previously 5. Briefly, 3 weeks old rats were anesthetized with ethyl ether and decapitated, and whole hippocampus was removed from the brain. Coronal brain slices (350 μm thickness) were cut using a vibrantly blade microtome in ice-cold artificial CSF (ACSF) containing the following (in mM) 126 NaCl, 2.5 KCl, 1 MgCl2, 1 CaCl2, 1.25 KH2PO4, 26 NaHCO3, and 20 glucose. ACSF was bubbled continuously with carbogen (95% O2 and 5% CO2) to adjust the pH to 7.4. Fresh slices were incubated in chamber with carbogenated ACSF and recovered at 34° C. for at least 1.5 h before they were transferred to recording chamber. For the recording of EPSCs, the individual slices were transferred to a recording chamber, and CA1 pyramidal neurons were viewed under upright microscopy (Olympus). The recording chamber (volume, 1.5 ml) was perfused at a rate of 4 ml min-1, with an external recording solution that contained the following (in mM): 119 NaCl, 26 NaHCO3, 2.5 KCl, 1 NaH2PO4, 1.3 MgCl2, 4 CaCl2, and 25 glucose, bubbled with 95% O2 and 5% CO2 (300-310 mOsm). Excitatory postsynaptic responses of CA1 pyramidal neurons were evoked by stimulating the Schaffer fibers through a constant-current pulse delivered by a bipolar tungsten electrode and recorded with Axopatch-200B amplifier (Molecular Devices). All above recordings were conducted in low-Mg²⁺ (0.25 mM) ACSF containing the GABAA blocker BMI (10 μM), the AMPAR blocker NBQX (5 μM) and ZL006 (1.0 μM). Stimulating electrode was placed in CA1 stratum radiatum at least 60-80 μm away from the cell body layer. The current intensity of test stimuli (25-50 μA) was set to produce half-maximal EPSPs. The baseline was recorded at least 10 min to ensure the stability of the response. Data were collected with pClamp 9.2 software and analyzed using Clampfit 9.2 (Molecular Devices). ZL006 does not inhibit NMDARs EPSCs, suggesting that ZL006 does not inhibit NMDA receptor conductance.

### EXAMPLE

Social Interaction. Adult male rats are evaluated in a conventional social interaction assay. Compounds described herein are evaluated compared to vehicle controls. Animals are observed for duration of time spent in regions of open field test (inner region versus outer region) and line crossings during a 60 min period following a systemic injection of vehicle or a test compound.

### EXAMPLE

Open Field Test of Anxiety. Adult male rats are evaluated in a conventional open field test. Compounds described herein are evaluated compared to vehicle controls. Animals are observed for duration of time spent in regions of the open field (inner region versus outer region) and line crossings during a 60 min period following a systemic injection of vehicle or a test compound.

### EXAMPLE

Results. Animals are systemically pre-treated by i.p. injections with two different agents that are known to disrupt the nNOS-PSD95 interaction, IC87201 (4 mg/kg) and ZL006 (10 mg/kg), 60 min prior to a 5 min open field test immediately followed by a 5 min social interaction test. In the open field test, there was not observed a significant difference between the duration of time spent in regions of the open field (inner region versus outer region) nor in the number of line crossings when comparing any of the test compound treatment groups and the vehicle-treated control group indicating that the compounds described herein do not cause anxiety side effects. (Inner time F(2,17)=0.4, p=0.678; Outer time F(2,17)=0.4, p=0.678; Line crossings F(2,17)=1.0, p=0.405; n=6/group). In addition, there was not observed a significant difference between ZL006 and the vehicle-treated control group in the social interaction assay, again indicating that the compounds described herein do not cause anxiety side effects. The group treated with IC87201 showed an improvement in social interaction duration (F(2,17)=6.6, p=0.009; n=6/group; Dunnett's test 2 tailed).

### EXAMPLE

Morris Water Maze. Adult male rats are evaluated in a conventional Morris water maze assay. Compounds described herein are evaluated compared to vehicle controls. Animals are observed for memory in a Morris Water Maze for distance travelled before finding the platform, and latency to find a platform.

A circular swimming pool (Jiliang Neuroscience Inc.) measuring 138 cm in diameter and 45 cm in height was filled with opaque water made by white nontoxic paint to a depth of 33 cm at 24±2° C. Four starting points around the edge of the pool were designated as N, E, S, and W, which divided the pool into four quadrants. A platform, 6 cm in diameter, was located in a constant position in the middle of one quadrant. To render it invisible to the mice, platform was submerged 1.2 cm below the surface of the water, which was invisible to the mice. The task for the mice was to escape from the water by locating the hidden platform. Two days before the start of training, the mice were given a pre-training session in which they were allowed to swim freely in a water tank for 60 s without an escape platform. One block of four trails was given for six consecutive days. On all 6 d, mice were injected i.v. with 3 mg kg-1 of ZL006 or vehicle 30 min before their first trial. For each trial, the mouse was placed in the water facing the wall of the pool at one of four starting points and allowed to swim for a maximum of 90 s. If the mice found the platform, they were allowed to remain on it for 10 s; the mice not finding the platform were guided to it and allowed to remain there for 10 s. Each trial was videotaped via a ceiling-mounted video camera and the animal's movement was tracked using Ethovision 24 software (Noldus Information Technology), which allows the calculation of various measures such as latency (time to reach the platform) and swimming speed. At days 7, mice were given 60 s retention probe test in which the platform was removed from the pool. During retention, the number of crossings of the platform location and the time spent in the target quadrant were measured. All Morris water maze tests were performed between 08:00 and 12:00 AM.

Software (Noldus Information technology), which allows the calculation of various measures such as latency (time to reach the patform) and swimming speed. At days 7, mice were given 60 s retention probe test in which the platform was removed form the pool. During retention, the number of crossings of the platform location and the time spent in the target quadrant were measured. All Morris water maze tests were performed between 08:00 and 12:00 AM.

There was not observed a significant difference between the distance travelled before finding the platform (F(3,21)=0.6, p=0.632; n=5-6), nor latency to find a platform (F(3,21)=0.4, p=0.726; n=5-6), when comparing any of the test compound treatment groups and the vehicle-treated control group indicating that the compounds described herein do not cause adverse cognitive side effects.

### EXAMPLE

NMDA-induced increase in cGMP in primary rat hippocampal neurons Neonatal rat hippocampal cultures were prepared according to Brewer (1997). Cells were cultured for 14-21 days before testing. NMDA (100 mM final) increased cGMP (measured by a cGMP-RIA kit from Perkin-Elmer) within 2-15 min of addition. The NMDA receptor antagonist, MK-801, and a NOS catalytic inhibitor, L-NAME, were used as positive controls. IC87201 attenuates the NMDA-induced increase of cGMP in primary cultured hippocampal neurons dose-dependently attenuated NMDA-induced increases in cGMP, an indirect measurement of nitric oxide production.

### EXAMPLE

Cell viability assays. An LDH release assay was used for the measurement of cell viability. Cortical neurons were stimulated with glutamate and glycine in Mg²⁺-free Locke's buffer. The neurons were washed with the buffer and incubated in cell-culture media for 12 h. Subsequently, LDH in the cell-culture media and total LDH after cell lysis were measured according to the manufacturer's instructions. LDH release was defined as ratio of LDH in the media to total LDH and normalized to the fold of control.

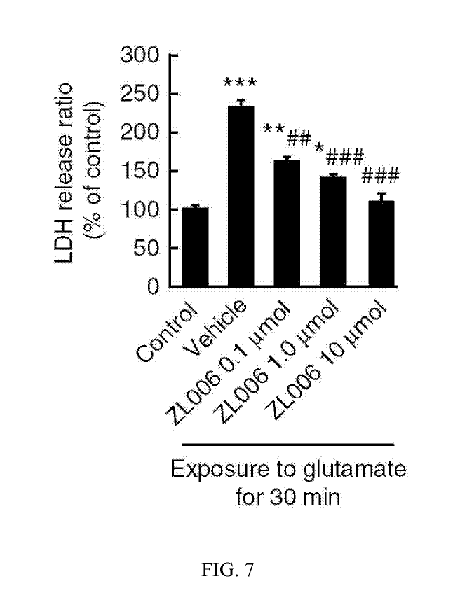

IC87201 and ZL006 disrupt nNOS downstream signaling as measured in glutamate receptor induced cell death in primary neuronal slices, as shown in FIG. 7.

### EXAMPLE

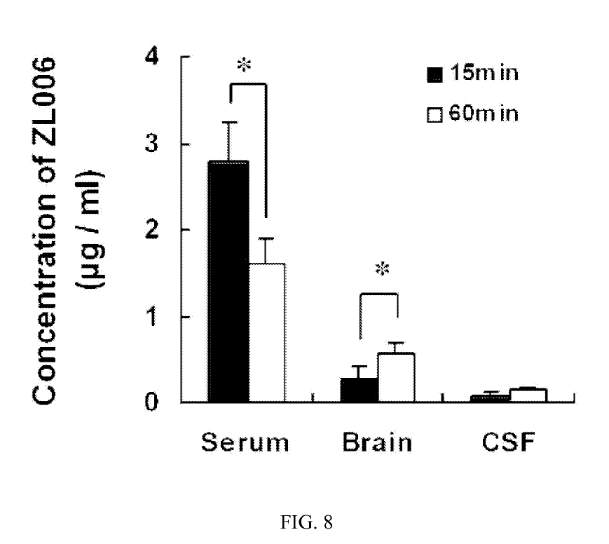

CNS level after systemic administration (Zhou 2010). Analysis of ZL006 concentrations in serum, CSF and brain tissue. For drugs that directly act on targets in the central nervous system (CNS), it is believed that sufficient drug delivery into the brain is a prerequisite for efficient drug action. Systemically administered drugs can reach CNS by passage across the endothelium of capillary vasculatures, the so-called blood-brain barrier (BBB). Cerebrospinal fluid (CSF) can be used as a useful surrogate for in vivo assessment of CNS exposure and provides an important basis for the selection of drug candidates for entry into development. Concentrations of ZL006 (1.5 mg/kg, i.v.) in serum, brain tissue and CSF were measured at 15 and 60 min after the dosing. Blood was withdrawn through the common carotid artery. Then, rats were sacrificed by decapitation and the brain tissue was rapidly removed, rinsed with cold saline and weighed. For drug concentration measurement in CSF, rats were anesthetized by chloral hydrate anesthesia (350 mg/kg, i.p.) 15 min before CSF was taken and 50 to 100 μl of CSF were taken by cisterna magna puncture at 15 or 60 min after the dosing. Serum, CSF and total brain tissue concentrations were determined using the HPLC method with ultraviolet detection. Chromatographic separation was performed using a reversed-phase (C18) stainless steel column. The mobile phase consisted of methanol-aqueous 30 mM HAc (54:45, v/v). The flow-rate was set at 1.0 ml min₋₁, and the sample size was fixed at 20 μl. The column temperature was maintained at 30° C. Wavelength was set at 284 nm. A concentrated stock solution of ZL006 (0.56 mg/ml) was prepared in methanol and was further diluted into 0.0219-5.6 μg/ml with serum, CSF or the supernatant from brain homogenate for the preparation of standard samples. All the solutions were stored at 4° C. For the analysis, serum (100 μl), CSF (50 μl) or the supernatant from brain homogenate (100 μl) and shaken on a vortex mixer for 3 min. After centrifuging at 10,000 rpm for 10 min, 20 μl of the supernatant liquid was injected into the HPLC system for analysis. ZL006 shows significant penetration in the CNS, as shown in FIG. 8.

### EXAMPLE

Generation of fusion proteins. nNOS (a.a. 1-299, encoding the PSD95 binding domain) was generated by inserting human nNOS residues 1-299 into pGEX 4T3 such that the clone was in frame with the glutathione S-transferase (GST) coding sequence of the vector. This ‘GST-nNOS’ was expressed in bacteria, and purified using glutathione Sepharose chromatography and thrombin cleavage, eluting purified nNOS 1-299 protein. This nNOS (1-299) was used in the in vitro binding assay. The protein sequence for human nNOS (1-299) is 94% and 96% homologous to mouse and rat nNOS (1-299) respectively.

### EXAMPLE

Tat-nNOS (1-299) fusion protein was generated by insertion of human nNOS residues 1-299 into a pRSET-B vector containing the coding sequence for the protein-transduction domain (YGRKKRRQRRR) of HIV-1 Tat protein. This tat-nNOS fusion contained a Tat-sequence and either a 6¥ or 10¥ His-tag at its N-terminal. Tat-nNOS fusion protein was expressed in bacteria, purified under denaturing conditions on a nickel-nitrilotriacetic acid (NTA) column and dialyzed against 1¥ calcium and magnesium-free phosphate buffered saline (PBS) before use. For a negative control, a non-transducing tat-nNOS containing the sequence (KALGISYGRKK) of Tat protein and the same 1-299 residues of nNOS was used.

### EXAMPLE

PSD95 containing PDZ domains 1-3 (residues 1-435, based on the human PSD95 sequence) was subcloned into a biotin expression plasmid such that the coding sequence was in frame with the biotin acceptor peptide. The fusion protein was expressed in bacteria in the presence of biotin and purified using streptavidin affinity chromatography. The purified protein contained biotin at its internal biotin acceptor site and is referred to as biotinylated PSD95. This was used in the in vitro binding assay. PDZ domain two of PSD95 is mainly responsible for the binding PSD95 to nNOS (Cho et al., 1992) and this protein sequence is 100% identical between human, rat and mouse.

### EXAMPLE

In vitro nNOS-PSD95 interaction assay. Recombinant nNOS (a.a. 1-299, 5 mg·mL-1), cleaved from GST-nNOS, was used to coat wells of an Immulon 96-well plate. After blocking non-specific sites with SEA block (Pierce) and further washing, biotinylated PSD95 (12.5 nM) was added as ligand' and binding continued for 2 h at room temperature before the reaction was terminated by repeated washing with PBS containing 0.05% Tween 20. The biotinylated PSD95-nNOS complex was detected by streptavidin-europium (Perkin-Elmer, Waltham, Mass., USA). After release of the europium by an enhancement solution (Perkin-Elmer), the increased fluorescence was measured using a DELFIA research fluorimeter (Perkin-Elmer). Using this in vitro binding assay, a high throughput screen was carried out to identify small molecule inhibitors which can disrupt the protein-protein interaction between nNOS and PSD95 Inhibitors were then tested for their ability to block the NMDA-induced increase in cGMP production (an indirect measurement of NO production) in neuronal cultures. One lead compound was identified to have efficacy in cell-based assays and was modified to improve stability. This modified small molecule, IC87201, 2-((1H-benzo[d][1,2,3]trizol-5-ylamino)methyl)-4-6-dichlorophenol (FIG. 1), was further characterized.

### EXAMPLE

nNOS enzymatic assay. nNOS was partially purified from frozen rat brain (Pel-Freez, Rogers, Ariz., USA) supernatant using 2!,5!-ADP sepharose and calmodulin-sepharose chromatography (both from Pharmacia Biotech, Piscataway, N.J., USA), according to Schmidt et al. (1991). The final pooled fractions, in 50 mMTris,pH7.5, 2 mM dithiothreitol (DTT), 1 M NaCl, 10% glycerol and 5 mM ethylene glycol tetraacetic acid (EGTA), were collected, concentrated and frozen. nNOS enzymatic activity was measured by the conversion of oxyhaemoglobin to methaemoglobin byNO essentially as described in Dawson and Knowles (1998). All buffers, inhibitors and equipment were prewarmed to 37° C. prior to assay. The final concentration of the components in the assay were 50 mM HEPES, pH 7.4, 100 mM DTT, 1 mM CaCl2, 5 mM oxyhaemoglobin, 12 mM tetrahydrobiopterin (BH4), 120 mM NADPH, 1 mM FMN, 1 mM FAD and 0.1 mM calmodulin. The reaction was initiated by the addition of partially purified rat brain nNOS. After quick mixing, the reaction absorbance was continuously measured at 405 nM and 420 nM for 30-60 min (at 10-20 s intervals) on a SpectraMax 250 reader (Molecular Devices, Sunnyvale, Calif., USA). L-NGmonomethyl arginine citrate was used as a positive control.

### EXAMPLE

NMDA-induced increase in cGMP in primary rat hippocampal neurons. Neonatal rat hippocampal cultures were prepared according to Brewer (1997). Cells were cultured for 14-21 days before testing. NMDA (100 mM final) increased cGMP (measured by a cGMP-RIA kit from Perkin-Elmer) within 2-15 min of addition. The NMDA receptor antagonist, MK-801, and a NOS catalytic inhibitor, L-NAME, were used as positive controls.

### EXAMPLE

Compound preparation testing. IC87201, 2-((1H-benzo[d][1,2,3]triazol-5-ylamino)methyl)-4,6-dichlorophenol, was synthesized by standard reductive amination of appropriate amines and aldehydes. For in vitro assays, IC87201 was prepared in 100% DMSO, then diluted into PBS and 0.1% bovine serum albumin (BSA). For cellbased assays, IC87201 was prepared in 100% DMSO and then diluted into control saline solution (120 mM NaCl, 5.4 mM KCl, 1.8 mM CaCl2, 25 mM Tris-HCl, 15 mM glucose, pH 7.5). For mouse experiments, IC87201 was prepared from a stock solution of 20 mM in 50% DMSO/50% 0.9% saline. This stock was then diluted to appropriate concentrations with a final DMSO concentration of 5% or less. Injection volume was 5 mL for i.t. administration. For i.p. administration, the injection volume was 100 mL. For rat models, IC87201 was dissolved in 20% DMSO in PBS, then 10 mL was administered through an i.t. catheter. Tat-nNOS and nt-tat-nNOS were purified as described above and dialyzed into 0.9% saline or PBS. In all cases, studies were vehicle matched.

### EXAMPLE

NMDA-induced nociceptive behavioural responses. Intrathecal administration of NMDA (0.3 nmol) produced scratching and biting responses in the first minute after injection (Aanonsen and Wilcox, 1987). This NMDA-induced scratching behaviour is blocked by NMDA receptor antagonists, but not by NOS catalytic inhibitors (Roberts et al., 2005), thus, it appears to be NO independent.

### EXAMPLE

Rotarod assay of sedation/motor impairment. After two training sessions, mice were placed for 300 s on an accelerating (4-40 rpm) rotarod (Ugo Basile, Varese, Italy).We measured the latency to fall before and after delivery of buffer, IC87201, tat-nNOS or control tat-nNOS. Data are presented as the time (seconds) the mice stayed on the rotarod (with 300 s as the maximum) or by the formula: % motor impairment=(pre-drug latency−post-drug latency)/(pre-drug latency ¥100%) (Fairbanks et al., 2000). In this latter case, mice that walked for 300 s would have a motor impairment of 0%.

### EXAMPLE

Spinal catheter implantation and the CCI model. Adult male Sprague-Dawley rats (Charles Rivers; 350-450 g) were anaesthetized with ketamine and medetomidine (75 mg·kg-1, 25 mg·kg-1, i.p.). The spinal catheter was implanted using the method of Yaksh and Rudy (1976) so that the distal end of the catheter extended to the lumbar enlargement. CCI was then performed on the left sciatic nerve trunk as described by Bennett and Xie (1988). Animals were given atipamezole (25 mg·kg-1, i.p.) post-surgery and monitored until recovery from anaesthesia. Animals were tested for signs of motor impairment 1 day post-surgery and those with impaired motor function were excluded from further experimentation. Spinal catheters were flushed with 10 mL of sterile saline each post-operative day with the exception of drug testing day.

1. A method for treating PTSD or a related disease in a host animal, the method comprising the step of administering to the host animal a therapeutically effective amount of a composition comprising one or more modulators of NMDA-PSD95-nNOS signaling.

2-3. (canceled)

4. The method of claim 1 wherein at least one modulator is an NMDA NR2B receptor antagonist.

5. The method of claim 1 wherein at least one modulator is compound of formula 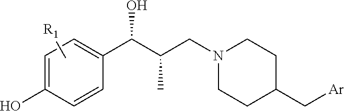
or a pharmaceutically acceptable salt thereof, wherein
Ar is optionally substituted aryl or heteroaryl; and
R₁ represents from 0 to 2 substituents independently selected from the group consisting of amino, hydroxyl, halo, thiol, alkyl, haloalkyl, heteroalkyl, nitro, sulfonic acids and derivatives thereof, and carboxylic acids and derivatives thereof.

6. The method of claim 5 wherein Ar is optionally substituted phenyl.

7. The method of claim 6 wherein Ar is phenyl.

8. (canceled)

9. The method of claim 1 wherein the modulator is compound Ro25-6981.

10. (canceled)

11. The method of claim 1 wherein the modulator is Tat-NR2B9c.

12. (canceled)

13. The method of claim 1 wherein at least one modulator is a compound of the formula 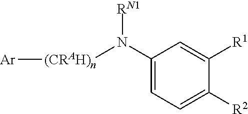
or a pharmaceutically acceptable salt thereof, wherein:
one of R¹ and R² is carboxylic acid or a derivative thereof, and the other is hydroxy or a derivative thereof.
Ar is optionally substituted aryl or optionally substituted heteroaryl;
R^{A} is independently selected in each instance H or alkyl;
R^{N1} is H, acyl, or a nitrogen prodrug forming group, or alkyl, cycloalkyl, heteroalkyl, cycloheteroalkyl, arylalkyl, or heteroarylalkyl, each of which is optionally substituted; and
n is an integer from 1 to about 4.

14. (canceled)

15. The method of claim 13 wherein one of R¹ and R² is carboxylic acid, and the other is hydroxy.

16. The method of claim 13 wherein R¹ is OH or OMe.

17. The method of claim 13 wherein R² is CO₂H or CO₂Me.

18. The method of claim 1 wherein at least one modulator is a compound of the formula 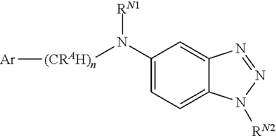
or a pharmaceutically acceptable salt thereof, wherein:
Ar is optionally substituted aryl or optionally substituted heteroaryl;
R^{A} is independently selected in each instance H or alkyl;
R^{N1} and R^{N2} are each independently selected in each instance from the group consisting of H, acyl, and nitrogen prodrug forming groups, and alkyl, cycloalkyl, heteroalkyl, cycloheteroalkyl, arylalkyl, and heteroarylalkyl, each of which is optionally substituted; and
n is an integer from 1 to about 4.

19. The method of claim 18 wherein Ar is optionally substituted aryl.

20. The method of claim 18 wherein Ar is 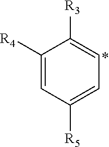
where * indicates the point of attachment;
R³ is hydrogen, hydroxy, or methoxy; and
R⁴ and R⁵ are independently selected from the group consisting of hydrogen, fluoro, chloro, or bromo.

21-34. (canceled)

35. The method of claim 1 wherein at least one modulator is a compound of the formula 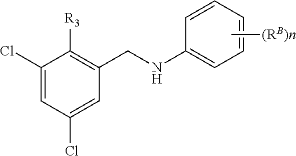
or a pharmaceutically acceptable salt thereof, wherein
R^{B}, independently, is selected from the group consisting of C₁₋₄alkyl, halo, CF₃, OCF₃, C(═O)R^{a}, C(═O)OR^{a}, N(R^{a})₂, C(═O)N(R^{a})₂, NR^{a}C(═O)N(R^{a})₂, OR^{a}, SR^{a}, NO₂, CN, SO₂N(R^{a})₂, SOR^{a}, SO₂R^{a}, and OSO₂CF₃; or two R¹ groups can be taken together with the carbon atoms to which they are attached to form an optionally substituted 5- to 7-membered aliphatic or aromatic ring, and optionally containing one to three heteroatoms selected from the group consisting of oxygen, nitrogen, and sulfur
R³is hydrogen or OH;
R^{a}, independently, is selected from the group consisting of hydro, C₁₋₄alkyl, aryl, and heteroaryl; and
n is an integer 0 through 4.

36. The method of claim 35 wherein two R^{B} groups are taken together to form a 5- or 6-membered heteroaryl group selected from the group consisting of

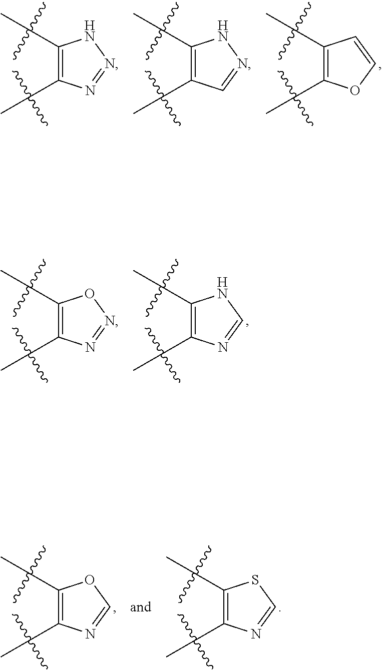

37. The method of claim 35 wherein two R^{B} groups are taken together, with the phenyl ring to which they are attached, to form a bicyclic aromatic ring system selected from the group consisting of, naphthalene, indene, benzoxazole, benzothiazole, benzisoxazole, benzimidazole, quinoline, indole, benzothiophene, and benzofuran, or two R¹ groups are taken together to form 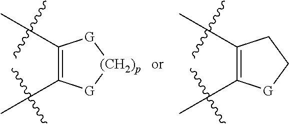
where p is 1 or 2: and G, independently, is C(R^{a})₂, O, S, or NR^{a}.

38-40. (canceled)

41. The method of claim 1 wherein at least one modulator is a tetrapeptide or a pentapeptide of the formula
A-B-C-D-E
or a pharmaceutically acceptable salt thereof;
wherein A is absent, or A is Pro or Val; B is Glu, Gln, or Arg; C is Thr; D is Asp, Asn, or His; and E is Val, Leu, or Ile; or
wherein B is Asp when D is Glu; and
where the terminal NH₂ is optionally acylated, such as acetylated, or optionally linked to Tat.

42. The method of claim 41 wherein at least one modulator is a peptide selected from the group consisting of RQIKIWFQNRRMKWKKNAKAVETDV (SEQ. ID. NO. 1), RQIKIWFQNRRMKWKKAVEATA (SEQ. ID. NO. 2), KNAKAVEDTA (SEQ. ID. NO. 3), KAVEDTA (SEQ. ID. NO. 4), NAKAVETDV (SEQ. ID. NO. 5), VETDV (SEQ. ID. NO. 6), VEDTV (SEQ. ID. NO. 7), VETDV-amide (SEQ. ID. NO. 8), acetyl-VETDV (SEQ. ID. NO. 9), Tat-VETDV (SEQ. ID. NO. 10), PETDV (SEQ. ID. NO. 11), VQTDV (SEQ. ID. NO. 12), VDTDV (SEQ. ID. NO. 13), VRTDV (SEQ. ID. NO. 14), VKTDV (SEQ. ID. NO. 15), VEVDV (SEQ. ID. NO. 16), VESDV (SEQ. ID. NO. 17), VETNV (SEQ. ID. NO. 18), VQTNV (SEQ. ID. NO. 19), VETLV (SEQ. ID. NO. 20), VETEV (SEQ. ID. NO. 21), VDTEV (SEQ. ID. NO. 22), VETHV (SEQ. ID. NO. 23), VETDL (SEQ. ID. NO. 24), VETDI (SEQ. ID. NO. 25), VETDG (SEQ. ID. NO. 26), VETDA (SEQ. ID. NO. 27), and ETDV (SEQ. ID. NO. 28).

43-63. (canceled)


SEQUENCE LISTING (29 sequences)

SEQ ID NO: 1 - protein, 25 aa, Homo sapiens
RQIKIWFQNRRMKWKKNAKAVETDV

SEQ ID NO: 2 - protein, 22 aa, Homo sapiens
RQIKIWFQNRRMKWKKAVEATA

SEQ ID NO: 3 - protein, 10 aa, Homo sapiens
KNAKAVEDTA

SEQ ID NO: 4 - protein, 7 aa, Homo sapiens
KAVEDTA

SEQ ID NO: 5 - protein, 9 aa, Homo sapiens
NAKAVETDV

SEQ ID NO: 6 - protein, 5 aa, Homo sapiens
VETDV

SEQ ID NO: 7 - protein, 5 aa, Homo sapiens
VEDTV

SEQ ID NO: 8 - protein, 5 aa, Homo sapiens
  MISC_FEATURE (5)..(5): The amino acid at position 5 is valine amide
VETDX

SEQ ID NO: 9 - protein, 5 aa, Homo sapiens
  MISC_FEATURE (1)..(1): The amino acid at position 1 is acetyl valine
XETDV

SEQ ID NO: 10 - protein, 16 aa, Homo sapiens
YGRKKRRQRRRVETDV

SEQ ID NO: 11 - protein, 5 aa, Homo sapiens
PETDV

SEQ ID NO: 12 - protein, 5 aa, Homo sapiens
VQTDV

SEQ ID NO: 13 - protein, 5 aa, Homo sapiens
VDTDV

SEQ ID NO: 14 - protein, 5 aa, Homo sapiens
VRTDV

SEQ ID NO: 15 - protein, 5 aa, Homo sapiens
VKTDV

SEQ ID NO: 16 - protein, 5 aa, Homo sapiens
VEVDV

SEQ ID NO: 17 - protein, 5 aa, Homo sapiens
VESDV

SEQ ID NO: 18 - protein, 5 aa, Homo sapiens
VETNV

SEQ ID NO: 19 - protein, 5 aa, Homo sapiens
VQTNV

SEQ ID NO: 20 - protein, 5 aa, Homo sapiens
VETLV

SEQ ID NO: 21 - protein, 5 aa, Homo sapiens
VETEV

SEQ ID NO: 22 - protein, 5 aa, Homo sapiens
VDTEV

SEQ ID NO: 23 - protein, 5 aa, Homo sapiens
VETHV

SEQ ID NO: 24 - protein, 5 aa, Homo sapiens
VETDL

SEQ ID NO: 25 - protein, 5 aa, Homo sapiens
VETDI

SEQ ID NO: 26 - protein, 5 aa, Homo sapiens
VETDG

SEQ ID NO: 27 - protein, 5 aa, Homo sapiens
VETDA

SEQ ID NO: 28 - protein, 4 aa, Homo sapiens
ETDV

SEQ ID NO: 29 - protein, 11 aa, Homo sapiens
YGRKKRRQRRR